(19) United States
(12) Patent Application Publication
(10) Pub. No.: US 2021/0005305 A1
(43) Pub. Date: Jan. 7, 2021

(54) METHOD AND APPARATUS FOR REHABILITATION TRAINING OF COGNITIVE FUNCTION

(71) Applicant: AIMMED CO., LTD., Seoul (KR)

(72) Inventors: Young Joon LEE, Seoul (KR); Young Jin YOO, Osan-si (KR); Eun Young KIM, Seoul (KR); Tae Kwon KIM, Seoul (KR); Seung Hoon NAM, Yongin-si (KR)

(21) Appl. No.: 16/977,805

(22) PCT Filed: Nov. 29, 2018
(86) PCT No.: PCT/KR2018/014155

§ 371 Date: Sep. 2, 2020

(30) Foreign Application Priority Data
Mar. 26, 2018 (KR) 10-2018-0034597

(51) Int. Cl. G16H 20/70; A61B 5/16; A61B 5/00; A61B 5/12; G09B 19/00

(52) U.S. Cl. CPC G16H 20/70; A61B 5/163; A61B 5/0048; A61B 2505/09; A61B 5/4088; A61B 5/4836; G09B 19/00; A61B 5/12

(57) ABSTRACT

Disclosed are a method and an apparatus for rehabilitation training of a cognitive function. A method for rehabilitation training of a cognitive function may comprise the steps of: performing a cognitive function test by a cognitive rehabilitation service server; receiving a cognitive function test result of the cognitive function test by the cognitive rehabilitation service server; determining a rehabilitation method matching the cognitive function test result, by the cognitive rehabilitation service server; and providing a user device with a rehabilitation content according to the rehabilitation method so as to perform rehabilitation training, by the cognitive rehabilitation service server.

### TECHNICAL FIELD

The present invention relates to rehabilitation training methods, and more specifically, to a method and device for rehabilitation training of a cognitive function.

### BACKGROUND ART

Most dementia screening tests currently rely on simple cognitive function tests such as Mini-Mental State Examination (MMSE), facing the following issues: 1) The test takes usually 15 minutes per person and lacks time efficiency; 2) These tests are paper-and-pencil type tests which may not be conducted on people with vision, hearing, or motion disabilities and are thus hard to apply to many elderly people; 3) The need for well-trained testers raises costs and, in some regions, it may be impossible to secure testers; and 4) A separate examination space is required because the tests are performed on a face-to-face basis between the tester and the testee.

To address the restrictions, computer-based or smart pad-based neurocognitive tests have been developed. Computer-based neurocognitive tests are suitable for early detection of cognitive changes in the elderly, minimizes floor and ceiling effects, provides a standardized format, and accurately records the accuracy and speed of response with moisture sensitivity that is impossible in standard management. These tests have the advantage of saving potential costs (material costs, consumables, and time required for test managers). They have the potential to screen large populations. Automated Neuropsychological Assessment Metrics (ANAM), Computer-Administered Neuropsychological Screen for Mild Cognitive Impairment (CANS-MCI), Cambridge Neuropsychological Test Automated Battery (CANTAB), CNS Vital Signs, Computerized Neuropsychological Test Battery (CNTB), Cognitive Drug Research Computerized Assessment System (COGDRAS-D), CogState, Cognitive Stability Index (CSI), MCI Screen (MCIS), MicroCog, and Mindstreams (Neurotrax) have been developed and commercially available as computer-based test tools. The National Center for Geriatrics and Gerontology functional assessment tool (NCGG-FAT) has been developed as an assessment tool for assessing multidimensional neurocognitive function using a tablet PC (personal computer). Presently, no smart pad-based test tools are commercially available in Korea.

### DETAILED DESCRIPTION OF THE INVENTION

### Technical Problem

An aspect of the present invention provides a cognitive function rehabilitation training method.

Another aspect of the present invention provides a device for performing a cognitive function rehabilitation training method.

### Technical Solution

According to an embodiment of the present invention, a method for rehabilitation of a cognitive function may comprise performing, by a cognitive rehabilitation service server, a cognitive function test, receiving, by the cognitive rehabilitation service server, a result of the cognitive function test, determining, by the cognitive rehabilitation service server, a rehabilitation method for the cognitive function test result, and providing, by the cognitive rehabilitation service server, rehabilitation content according to the rehabilitation method to a user device to perform rehabilitation training.

Meanwhile, reassessment on a user of the user device may be performed after the cognitive rehabilitation service server provides the rehabilitation content.

Further, the cognitive function test may be performed on at least one of an orientation area, a memory area, an attention concentration area, a visual perception area, and a language area.

Further, the cognitive function test result may include information about an assessment accuracy, an assessment time required, a user reaction time, and a score for each assessment area.

Further, the cognitive rehabilitation service server may detect a movement of an eye based on a gaze tracking module and track a position of a gaze to perform the cognitive function test and the rehabilitation training.

According to another embodiment of the present invention, a cognitive rehabilitation service server performing a cognitive function rehabilitation training method may include a processor. The processor may be configured to perform a cognitive function test, receive a result of the cognitive function test, determine a rehabilitation method for the cognitive function test result, and provide rehabilitation content according to the rehabilitation method to a user device to perform rehabilitation training.

Meanwhile, the processor may be configured to reassess a user of the user device after the processor provides the rehabilitation content.

Further, the cognitive function test may be performed on at least one of an orientation area, a memory area, an attention concentration area, a visual perception area, and a language area.

Further, the cognitive function test result may include information about an assessment accuracy, an assessment time required, a user reaction time, and a score for each assessment area.

Further, the processor may detect a movement of an eye based on a gaze tracking module and track a position of a gaze to perform the cognitive function test and the rehabilitation training.

### Advantageous Effects

According to embodiments of the present invention, the cognitive function rehabilitation training method and device may provide digital content using, e.g., speech recognition and gaze tracking technology per difficulty on each cognitive function item, e.g., memory/concentration/spatiotemporal ability to a cognitive function test and rehabilitation training system for people with low cognitive function (stroke, dementia, or mild cognitive impairment), slowing down or maintaining the reduction in cognitive function.

### BRIEF DESCRIPTION OF THE DRAWINGS

FIG. 1 is a concept view illustrating a cognitive rehabilitation system according to an embodiment of the present invention;

FIG. 2 is a concept view illustrating a cognitive function test and rehabilitation method according to an embodiment of the present invention;

FIG. 3 is a concept view illustrating a cognitive function test screen according to an embodiment of the present invention;

FIG. 4 is a concept view illustrating a screen for the results of a cognitive function test according to an embodiment of the present invention;

FIG. 5 is a concept view illustrating cognitive rehabilitation contents according to an embodiment of the present invention;

FIG. 6 is a concept view illustrating a cognitive function test and cognitive rehabilitation training method based on a gaze tracking technology according to an embodiment of the present invention;

FIG. 7 is a concept view illustrating a method for cognitive ability measurement and cognitive rehabilitation training based on speech recognition according to an embodiment of the present invention;

FIG. 8 is a concept view illustrating a cognitive rehabilitation training method according to an embodiment of the present invention; and

FIG. 9 is a concept view illustrating deep neural network analysis according to an embodiment of the present invention.

### DESCRIPTION OF EXEMPLARY EMBODIMENTS

The present invention is described below in detail in connection with particular embodiments thereof, taken in conjunction with the accompanying drawings. Embodiments are described thoroughly enough to make a skilled artisan practice the present invention. It should be noted that various embodiments of the present invention, although differing from each other, do not necessarily exclude each other. For example, specific shapes, structures, and characteristics described herein in connection with one embodiment may be implemented in other embodiments without departing from the spirit and scope of the present invention. It should also be appreciated that the location or arrangement of individual components in each embodiment may be varied without departing from the spirit and scope of the present invention. Thus, the following detailed description should not be intended as limiting, and the scope of the present invention is defined only by the appending claims and their equivalents so long as adequately described. The same reference denotations may be used to refer to the same or similar elements throughout the drawings and the specification.

Hereinafter, preferred embodiments of the present invention are described in detail with reference to the accompanying drawings.

According to an embodiment of the present invention, in a cognitive function rehabilitation training method, module-based cognitive function assessment and rehabilitation may be performed. Conventional mini mental state examination (MMSE)-based questionnaire formats are limited in use and less objective. However, a dialog-type configuration using a speech recognition-based module may minimize the patient's reluctance. Further, the cognitive function rehabilitation training method according to an embodiment of the present invention may assess patients with cognition dysfunction, who suffer from uncomfortable behavior or lowering in language ability, and enable rehabilitation training, using speech recognition and gaze tracking technology.

In other words, the cognitive function rehabilitation training method according to an embodiment of the present invention enables a cognitive function test considering users. The speech recognition-based assessment method is efficient because the illiteracy (incapable of reading, writing, and counting in everyday life) rate for elderly people ages 70 and above reaches 44.7%. In the case of assessment using a foreign program, a program suitable for domestic sentiment is needed because differences in culture and language may affect the accuracy of the test results. Thus, the present solution enables objective cognitive function assessment by efficiently gathering and analyzing data via sensor-based technology on the cognitive function assessment process which was possible only be skilled assessor.

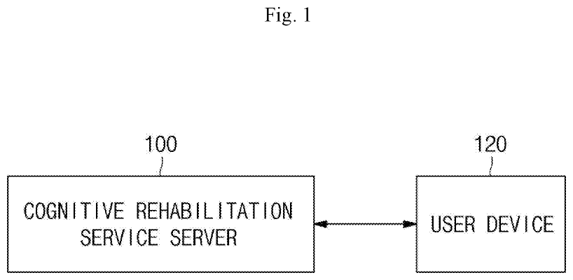

FIG. 1 is a concept view illustrating a cognitive rehabilitation system according to an embodiment of the present invention.

Referring to FIG. 1, a cognitive rehabilitation system for cognitive rehabilitation may include a cognitive rehabilitation service server 100 and a user device 120.

The cognitive rehabilitation service server 100 may provide cognitive rehabilitation test content for the user's cognitive rehabilitation, perform assessment on the cognitive rehabilitation test content, and provide cognitive rehabilitation content considering the results of assessment of the cognitive rehabilitation test content.

The user device 120 may receive the cognitive rehabilitation test content from the cognitive rehabilitation service server 100 and input an answer to the cognitive rehabilitation test content to the cognitive rehabilitation service server 100. Thereafter, the user device 120 may receive the cognitive rehabilitation content from the cognitive rehabilitation service server 100 and provide a cognitive rehabilitation training service to the user.

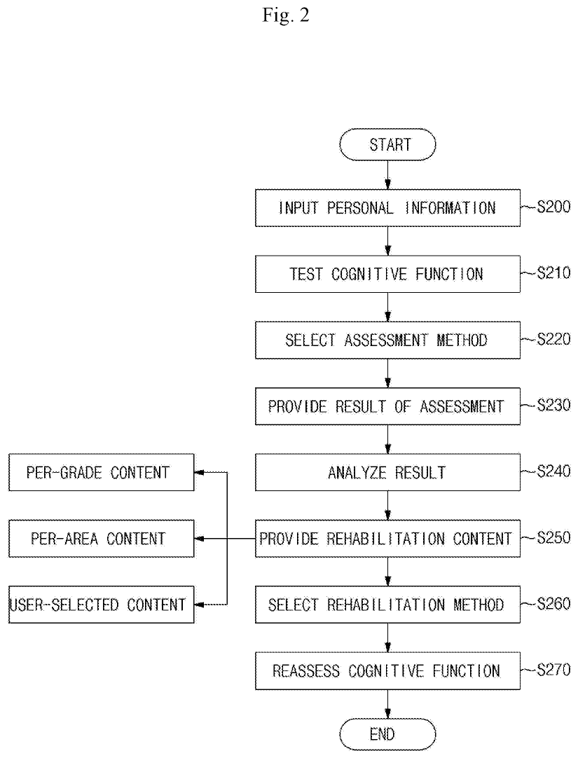

FIG. 2 is a concept view illustrating a cognitive function test and rehabilitation method according to an embodiment of the present invention.

FIG. 2 illustrates a method for assessing the user's cognitive function and, if the cognitive function is a predetermined threshold or less, providing the cognitive rehabilitation content as per the result of cognitive rehabilitation test to thereby perform rehabilitation for cognitive function.

Referring to FIG. 2, the user's personal information may be entered (step S200).

The user's personal information, such as gender, age, and name, may be entered.

The user's cognitive function test may be performed (step S210).

The assessment areas for the user's cognitive function may include one of orientation, memory, attention-concentration, visual perception, and language.

The results of the cognitive function test on the user are provided (step S220).

The results of assessment on the user's cognitive function may include accuracy for the cognitive function test, time required, response time, and score for each area.

Analysis for the user's cognitive function test results is provided (step S230).

Rehabilitation content is provided based on the analysis of the user's cognitive function test results (step S240).

The rehabilitation content may include content by grade, content by area, or user-selected content.

A rehabilitation method is selected (step S250).

The rehabilitation method is one of contents using the content by grade, content by area, or user-selected content.

Cognitive rehabilitation is performed based on the selected rehabilitation method (step S260).

After the cognitive rehabilitation, reassessment is performed on the cognitive function (step S270).

The reassessment of the user's cognitive function may regard orientation, memory, attention-concentration, visual perception, and language.

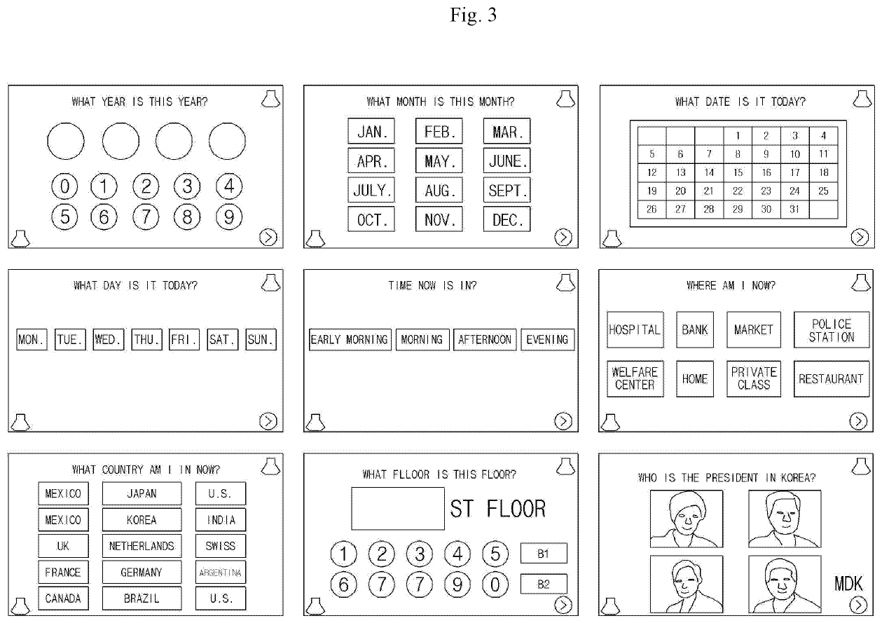

FIG. 3 is a concept view illustrating a cognitive function test screen according to an embodiment of the present invention.

FIG. 3 illustrates a cognitive function test screen provided to the patient.

Referring to FIG. 3, a questionnaire may be provided to the user via the cognitive function test screen.

The questionnaire provided to the user may be a questionnaire for checking the user's basic current cognitive state, such as current time (year, month, day, date, and hour) or common sense (country or president).

The questionnaire provided to the user may separately include a set of questions for each threshold age, and the set of questions may be provided in ascending order of difficulty considering the user's rate of correct answers. For example, a first question set may be a question set, for which the correct answer rate is 80% or more for age 8, a second question set may be a question set, for which the correct answer rate is 80% or more for age 9, and a third question set may be a question set, for which the correct answer rate is 80% or more for age 10. A determination of the user's cognitive level may be performed while the questionnaire is provided to the user in the order of the first question set, the second question set, and the third question set. Where the correct answer rate for an nth question set is a threshold or more, an n+2th question set may be provided to the user, with an n+1th question set skipped.

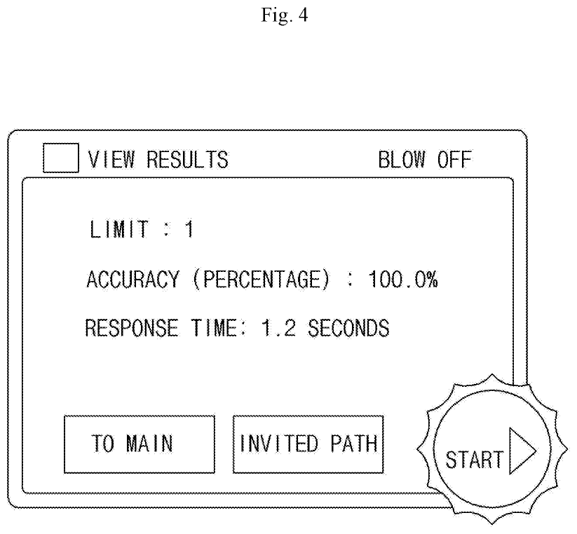

FIG. 4 is a concept view illustrating a screen for the results of a cognitive function test according to an embodiment of the present invention.

FIG. 4 illustrates a screen for the results of cognitive ability assessment.

Referring to FIG. 4, cognitive ability step information, cognitive ability assessment accuracy information, and cognitive ability assessment time information may be provided as the results of cognitive ability assessment.

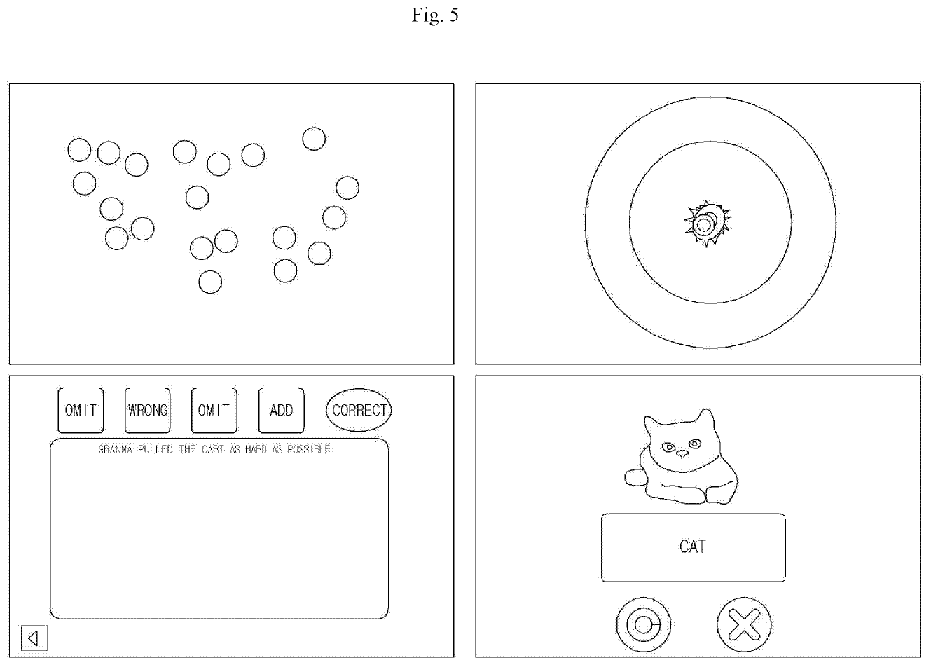

FIG. 5 is a concept view illustrating cognitive rehabilitation contents according to an embodiment of the present invention.

FIG. 5 discloses rehabilitation content for enhancing cognitive ability.

Referring to FIG. 5, rehabilitation content may be provided to enhance orientation, memory, attention-concentration, visual perception, and language ability.

The rehabilitation content may be divided into categories, such as memory, concentration, and visual perception ability and be given a score depending on the correct answer rate provided per difficulty. The rehabilitation content may be provided on a mobile application of the user device.

Specifically, memory training may be training for enhancing the ability of temporarily storing selected and entered information only while a task is performed or continuously storing it for a long time and outputting and utilizing it only when a relevant task is performed. The memory training may include location memory/figure memory/memory width training/story memory/plan memorizing/face memorizing/memory memorizing/procedure memorizing.

The visual perception training may be correction training for activating the ability of integrating and analyzing, in brain, the information entered via visual organizations from the external environment to thereby re-recognizing the target and enhance spatiotemporal interpretation ability. The visual perception training may include selecting the same picture, finding functions, matching names, finding the same pictures, finding the number of blocks, making shapes with blocks, and finding the position of a point.

The concentration training may be training to activate the active information processing procedure that selects a specific piece of information from among various pieces of information entered from the outside, retains the selected information only for a required time and turns the attention to a different target and then simultaneously selects two or more. The concentration training may include focus concentration training, counting training, same shape find training, spot find training, color match training, sound concentration training, counting training, draw-a-shape-with-dots training, selective concentration training, transformational concentration training, diachronic concentration training, continuous concentration training, and number-matching training.

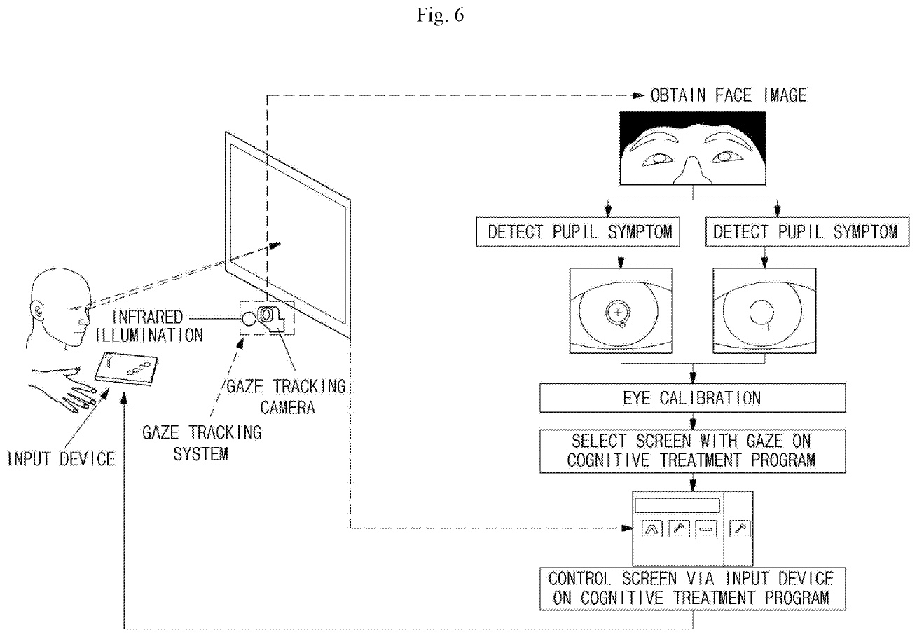

FIG. 6 is a concept view illustrating a cognitive function test and cognitive rehabilitation training method based on a gaze tracking technology according to an embodiment of the present invention.

FIG. 6 discloses a method for performing a cognitive function test and cognitive rehabilitation training based on gaze tracking technology.

Referring to FIG. 6, a gaze tracking module is a module for implementing gaze tracking technology which is the technology of detecting the eyes and tracking the gaze and be utilized as a user interface instead of touching, upon performing a cognitive function assessment and cognitive rehabilitation program.

A person with reduced cognitive ability due to, e.g., stroke, also suffers from a reduction in body function and, thus, the eye tracking module may be more useful than a touch-based interface.

A separate, table mount-type gaze tracking module may be used which may be attached to a smart device.

In the principle, brain stimulation-capable digital cognitive function rehabilitation content, such as of vision/voice, is utilized for people with reduced cognitive function to slow down the rate of cognitive decline.

Further, a user interface may be implemented based on speech synthesis technology. Measurement and rehabilitation training for cognitive ability may be performed based on text-to-speech (TTS) technology which may convert text information into such a natural speech as if a human speaks.

Specifically, questions or text may be spoken instead of, or together with, the text, upon performing the cognitive function assessment and cognitive function program on a smart device (e.g., a smartphone or tablet). A person with reduced cognitive ability due to, e.g., stroke, also suffers from a reduction in body function and, thus, this may be more useful than a touch-based interface.

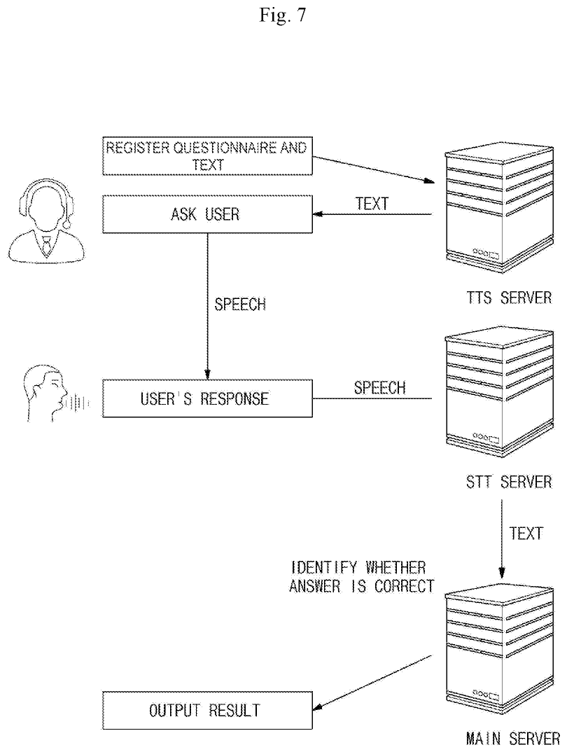

FIG. 7 is a concept view illustrating a method for cognitive ability measurement and cognitive rehabilitation training based on speech recognition according to an embodiment of the present invention.

FIG. 7 discloses a method for cognitive ability measurement and cognitive rehabilitation training based on a speech recognition function.

In a case where the user utters a speech via speech-to-text (STT) which converts a human speech into text, the speech may be converted into text and recognized and, based on the converted text, measurement for cognitive ability may be performed, and cognitive rehabilitation training may be carried out. Upon performing the cognitive ability test and cognitive rehabilitation program via a smart device (e.g., a smartphone or tablet), the user's answers are gathered instead of touch. Thus, this way may be more useful for people with lowered cognitive ability and body function, due to stroke, than touch-based interfaces. Many correct answers may be previously registered so that it may be determined whether the answers are correct answers to corresponding questions after listening to the user.

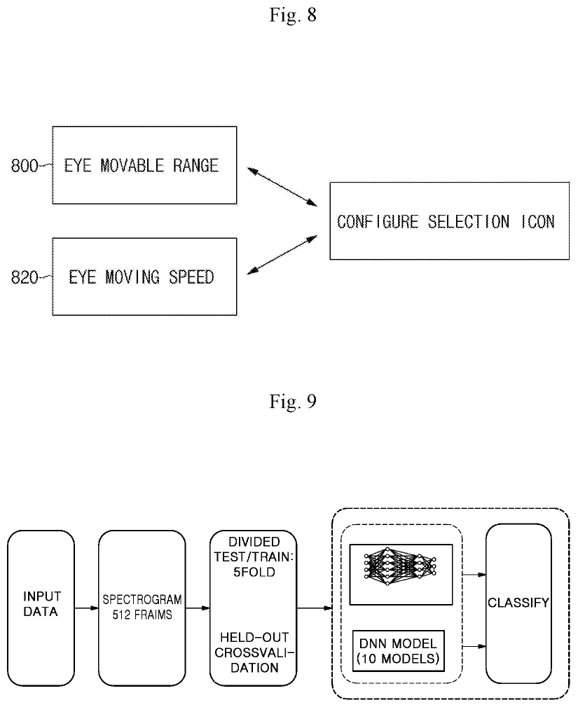

FIG. 8 is a concept view illustrating a cognitive rehabilitation training method according to an embodiment of the present invention.

FIG. 8 discloses a method for tracking the user's gaze for cognitive rehabilitation training.

Referring to FIG. 8, the gaze tracking module may previously configure the user interface considering the user's reaction rate and the range in which the user's eyes are movable in tracking the user's gaze.

For example, 10, as an average value, may be the average range in which the user's eyes are movable. The gaze tracking module may first determine the movable range 800 of the user's eyes to configure a gaze-based user interface. A range 800 in which the user's eyes are movable left/right/up/down may be determined.

Where the movable range 800 of the user's eyes is set, an icon indicating the user's selection may be moved on the user interface, considering the movable range 800. If the movable range 800 of the user's eyes is relatively smaller than the average movable range, the movement of the icon indicating the user's selection on the user interface, according to the movement of the user's eyes may relatively increase. In contrast, if the movable range 800 of the user's eyes is relatively larger than the average movable range, the movement of the icon indicating the user's selection on the user interface, according to the movement of the user's eyes may relatively decrease.

Further, configuration for the moving speed 820 of the user's eyes may also be performed. The speed at which the user may move his eyes conveniently may be measured and, thus, the moving speed of the selection icon may be varied as well. If the moving speed 820 of the user's eyes is relatively smaller than the average moving speed, the moving speed of the icon indicating the user's selection on the user interface, according to the movement of the user's eyes may relatively increase. In contrast, if the moving speed 820 of the user's eyes is relatively larger than the average moving speed, the moving speed of the icon indicating the user's selection on the user interface, according to the movement of the user's eyes may relatively decrease.

The cognitive rehabilitation service server may measure the movable range 800 of the user's eyes and the eye moving speed 820 and adaptively set the moving range and moving speed of the selection icon on the user interface according to the movable range 800 of the user's eyes and eye moving speed 820.

Further, according to an embodiment of the present invention, questions for a cognitive function test may be provided in various manners so as to more quickly and precisely perform the cognitive function test. Specifically, where the user's cognitive function is divided into a first step, a second step, . . . , an nth step, if assessment for the user's cognitive function is performed sequentially from the first step, the fatigue of assessment may be high.

Thus, for the user's cognitive function test, a first question set in which, starting from the middle, n/2th step, the lower steps (to the first step) and higher steps (to the nth step) are alternately mixed may be provided to the user. For example, if n is 10, the first question set may be configured in the order of the fifth step, fourth step, sixth step, third step, seventh step, second step, eighth step, first step, and tenth step. That is, from the middle step, its higher steps and lower steps may be alternately mixed.

Based on the distribution of the user's correct answers to the first question set, the user's first assessment may be performed. For example, where in the first question set, the correct answer rate in the first to sixth steps is not less than a first threshold (e.g., 80%), and the user's correct answer rate in the seventh to tenth steps is not more than a second threshold (e.g., 40%), a second question set for assessing the user's cognitive ability may be generated and provided from the sixth step to the first step. At this time, if the correct answer rate in the sixth step is not less than a third threshold (e.g., 70%), the questions in the sixth and higher step (e.g., the seventh step) may be provided to the user, and assessment for the user's cognitive function may be performed. In contrast, if the correct answer rate in the sixth step is less than the third threshold (e.g., 70%), the questions in the step (e.g., the fifth step) less than the sixth step may be provided to the user, and assessment for the user's cognitive function may be performed. In the same manner, questions may be provided to the user, based on the third threshold, so that less questions may be provided to the user, and assessment for the user's cognitive ability may be performed more efficiently and quickly. That is, given a reference step determined based on the correct answer rate for the first question set, the results of cognitive assessment on the reference step may be determined. Considering again the results of the cognitive assessment for the reference step, a transfer from the reference step to its relatively higher or lower step may proceed.

Such a manner makes it possible to efficiently provide a reduced number of questions to assess the user's cognitive ability in a simplified manner without the need for providing unnecessarily many questions for assessing the user's cognitive ability.

According to an embodiment of the present invention, candidate items for the development of a new neurocognitive screening tool for dementia may be extracted. In data banks tracked by the Korean Longitudinal Study on Cognitive Aging and Dementia (KLOSCAD) and database collected from the Department of Mental Health and Dementia Clinic, Seoul National University Hospital, Bundang, the neuropsychological test results of normal elderly people and elderly people with mild cognitive impairment and dementia are divided into a development data set and a validation data set and, then, the development data may be analyzed to constitute screening test items at the MMSE level.

The following table represents gather data items.

TABLE 1

| Serial number | No. |
|---|---|
| Demographic | Age |
| variables | Gender |
| | Education |
| Clinical assessment | Dementia diagnosis |
| | Depression scale score |
| | Severity of dementia |
| Neuropsychological | 0-15 seconds score |
| assessment score | 16-30 seconds score |
| (category fluency) | Early score |
| | 31-45 seconds score |
| | 46-60 seconds score |
| | Late score |
| | Number of perseverative responses |
| | Number of infiltration responses |
| | Conversion score |
| | Inefficient conversion score |
| | Category score |
| | Total score |
| Neuropsychological | First response high-frequency figure fitting count |
| assessment score | Second response high-frequency figure |
| (short version of | fitting count |
| Boston naming) | Third response high-frequency figure fitting count |
| | First response mid-frequency figure fitting count |
| | Second response mid-frequency figure fitting count |
| | Third response mid-frequency figure fitting count |
| | First response low-frequency figure fitting count |
| | Second response low-frequency figure fitting count |
| | Third response low-frequency figure fitting count |
| | First response final score |
| | Second response final score |
| | Third response final score |
| | High-frequency visual perception error |
| | High-frequency meaning-associated error |
| | High-frequency meaning-nonassociated error |
| | High-frequency phoneme error |
| | High-frequency DK |
| | High-frequency NR |
| | Mid-frequency visual perception error |
| | Mid-frequency meaning-associated error |
| | Mid-frequency meaning-nonassociated error |
| | Mid-frequency phoneme error |
| | Mid-frequency DK |
| | Mid-frequency NR |
| | Low-frequency visual perception error |
| | Low-frequency meaning-associated error |
| | Low-frequency meaning-nonassociated error |
| | Low-frequency phoneme error |
| | Low-frequency DK |
| | Low-frequency NR |
| Neuropsychological | Time orientation score |
| assessment score | Place orientation score |
| (MMSE-DS) | Memory registration score |
| | Attention concentration score |
| | Memory recall |
| | Naming score |
| | Shadowing score |
| | Third step command performing score |
| | Spatiotemporal constructional ability score |
| | Judge and understand score |
| | MMSE-DS total score |
| Neuropsychological | Perform 1 Infiltrated word count |
| assessment score | Perform 2 Infiltrated word count |
| (word list memory test) | Perform 3 Infiltrated word count |
| | Perform 1 Repeated word count |
| | Perform 2 Repeated word count |
| | Perform 3 Repeated word count |
| | Perform 1 Beginning word count |
| | Perform 2 Beginning word count |
| | Perform 3 Beginning word count |
| | Perform 1 Latest word count |
| | Perform 2 Latest word count |
| | Perform 3 Latest word count |
| | Perform 1 Matched word count |
| | Perform 2 Matched word count |
| | Perform 3 Matched word count |
| | Study score |
| | Word list memory test final score |
| | Beginning percentage |
| | Latest percentage |
| | Matching word count |
| Neuropsychological | Item 1 (circle) score |
| assessment score | Item 2 (diamond) score |
| (constructional | Item 3 (rectangle) score |
| behavior test) | Item 4 (cube) score |
| | Constructional behavior final score |
| | Closing-in |
| Neuropsychological | Infiltrated word count |
| assessment score | Repeated word count |
| (word list recall) | Word list recall final score |
| | Save rate |
| | Matching word count |
| | Non-matching word count |
| Neuropsychological | Word list recognition final score |
| assessment score | Response bias |
| (word list recognition) | |
| Neuropsychological | Item 1 (circle) recall score |
| assessment score | Item 2 (diamond) recall score |
| (constructional recall) | Item 3 (rectangle) recall score |
| | Item 4 (cube) recall score |
| | Constructional recall total score |
| | Constructional recall save rate |
| Neuropsychological | Constructional recognition final score |
| assessment score | Constructional recognition response bias |
| (constructional | |
| recognition) | |
| Neuropsychological | A final score (seconds) |
| assessment score | B final score (seconds) |
| (CLOX) (trail making) | Rate score |
| Neuropsychological | Memorize forward attention width |
| assessment score | Memorize backward attention width |
| (memorize number) | |
| Neuropsychological | FAB1 |
| assessment score | FAB2 |
| (FAB) | FAB3 |
| | FAB4 |
| | FAB5 |
| | FAB6 |
| | FAB total score |
| Neuropsychological | CLOX I score |
| assessment score | CLOX II score |
| (CLOX) | |

Further, according to an embodiment of the present invention, candidate items for the development of a new neurocognitive screening tool for dementia may be extracted and verified.

A pre-secured full data set is divided into a development data set and a validation data set, and the development data set may be used to extract neurocognitive pre-test items, and the validation data set may be used to assess the diagnosis accuracy of the mobile neurocognitive test constituted of the items extracted from the development data set.

Extraction of the candidate items may be performed by two methods: machine learning and traditional statistics modeling.

Machine learning is a method to extract an algorithm from data without a rule-based programming, and the statistics modeling is a method to formulate and model the relationships between variables in the form of mathematical formulas.

The type and amount of data already collected for this research is massive and includes many detailed examinations of the neuropsychological test, and there are many data dimensions. In analysis of such a high dimensionality-type data set, machine learning may be applied.

Further, a combination of pattern analysis and screening of test results for each patient group may be performed.

Further, according to an embodiment of the present invention, deep neural network (DNN) analysis may be performed.

FIG. 9 is a concept view illustrating deep neural network analysis according to an embodiment of the present invention.

Referring to FIG. 9, neural network is a scheme widely used in a pattern classification field, which trains features using a non-linear transfer function. The DNN using the same has a structure in which hidden layers between the input layer and the output layer are stacked one over another and is very effective in addressing issues with data of high-complicated dimension using an alternative algorithm that complements the shortcomings of the legacy artificial neural network model.

In the classification issue using deep learning, a most critical element lies in establishing a model that may represent the dementia group and normal group. In doing so, five representative models for cognitive function test are created for each of the dementia group and the normal group, using 10 or more test combinations, and it is presumed and assumed in the present invention that a different pattern is present for each model. Thus, when 10 models are established, and classification is performed per frame on the results of each test, more detailed classification may be performed than when two models (of dementia and normal) are established.

Deep neural network analysis using the ten models may provide the advantage of being able to classify test tools and result types more sensitive to diagnosis. A test for analyzing classification accuracy is designed to classify into 20 models and then finally determine the dementia group and normal group via a majority vote, and accuracy is analyzed once for every patient group by performing fivefold held-out cross validation five times.

Logistic regression analysis may be performed by traditional statistical modeling.

According to an embodiment of the present invention, a standardized coefficient (beta coefficient) is obtained using the logistics regression model to assess the relative criticality per test with the development of a diagnostic algorithm. A regression equation is configured using the calculated standardized coefficient, and a weighted composite score for each test characteristic is derived and is then used to find the test combination that represents the optimal diagnostic accuracy. At this time, the regression analysis may use stepwise regression and may be performed considering multi-collinearity.

According to an embodiment of the present invention, verification of the diagnostic algorithm may be performed. Reference validity is verified using the ANOVA for which the presence and absence of cognitive dysfunction is age-corrected based on golden standards, homogeneity validity is verified by the Pearson correlation test using MMSE, and cross validity is verified by bootstrapping or a jack-knife method, and the diagnostic accuracy may be analyzed using a receiver operator characteristic (ROC) analysis.

Further, according to an embodiment of the present invention, new dementia screening tool optimization may be carried out. A development database may be utilized to develop the optimal screening tool considering the convenience and diagnosis accuracy using some candidate test tool sets.

Validity verification of the new dementia screening tool may be performed. Validity of the developed dementia screening tool developed using the verification data set may be verified.

According to an embodiment of the present invention, cognitive rehabilitation training may be performed as follows. Factors influencing the difficulty of rehabilitation training include speed of presentation, time limit, number of simultaneous questions, complexity, and familiarity. That is, as the speed at which questions are presented increases, and the time of presentation decreases, the number of questions presented at the same time increases, and the questions become more unfamiliar and complicated, the difficulty increases. Depending on the difficulty, these factors may be varied, and other factors including the speed of presentation may be adjusted in the settings and each detailed content.

There are composed of one or more areas of concentration training, memory training, and orientation training and, in the case of a one-to-one matching scheme, the patient is allowed to respond with the O and X buttons in a touch/gaze tracking manner and to respond as “Yes” or “No” using a speech recognition scheme.

In a multiple-choice type, the patient is allowed to select a number or choose a correct one using an arrow using a touch/gaze tracking scheme or to say a number using a speech recognition scheme.

If the test for all the areas is done to provide the results of assessment, a result window is automatically displayed, and the total grade for accuracy and the average response time are presented, with per-area scores displayed as detailed information. Further, it is clearly represented using a graph whether the targeted person's cognitive level falls within a normal range or less than normal. The user may be recommended for proper content depending on the total score and per-area scores among the assessment results.

To utilize speech recognition upon cognitive function assessment and rehabilitation, the user interface enables mutual communication with the user and include a speaker for outputting speech signals and a microphone for receiving speech signals.

By a conversion step, the user's speech may be recognized and converted into text (speech-to-text (STT)), or the text may be converted into a speech (text-to-speech (TTS)). By a processing step, the converted test may be compared with a reference value pre-configured in the program to thereby determine whether the answer is correct or now. By a transmission step, the results of cognitive function assessment and rehabilitation are transmitted to the server.

The above-described method may be implemented as an application or in the form of program instructions executable through various computer components, which may then be recorded in a computer-readable recording medium. The computer-readable medium may include programming commands, data files, or data structures, alone or in combinations thereof.

The programming commands recorded in the computer-readable medium may be specially designed and configured for the present invention or may be known and available to one of ordinary skill in the computer software industry.

Examples of the computer readable recording medium may include, but is not limited to, magnetic media, such as hard disks, floppy disks or magnetic tapes, optical media, such as CD-ROMs or DVDs, magneto-optical media, such as floptical disks, memories, such as ROMs, RAMs, or flash memories, or other hardware devices specially configured to retain and execute programming commands.

Examples of the programming commands may include, but are not limited to, high-level language codes executable by a computer using, e.g., an interpreter, as well as machine language codes as created by a compiler. The above-described hardware devices may be configured to operate as one or more software modules to perform processing according to the present invention and vice versa.

While the present invention has been shown and described with reference to exemplary embodiments thereof, it will be apparent to those of ordinary skill in the art that various changes in form and detail may be made thereto without departing from the spirit and scope of the present invention as defined by the following claims.

1. A method for rehabilitation of a cognitive function, the method comprising:
performing, by a cognitive rehabilitation service server, a cognitive function test;
receiving, by the cognitive rehabilitation service server, a result of the cognitive function test;
determining, by the cognitive rehabilitation service server, a rehabilitation method for the cognitive function test result; and
providing, by the cognitive rehabilitation service server, rehabilitation content according to the rehabilitation method to a user device to perform rehabilitation training, wherein
the cognitive function test is performed based on touch recognition, speech recognition, or gaze tracking, wherein
the rehabilitation method is determined based on a method used for the cognitive function test, wherein
the cognitive rehabilitation service server detects a movement of an eye based on a gaze tracking module and tracks a position of a gaze to perform the cognitive function test and the rehabilitation training, and the cognitive rehabilitation service server detects the movement of the eye using the gaze tracking module and uses the detected eye movement, as a user interface, instead of a touch upon performing a cognitive function assessment and cognitive rehabilitation program, wherein
the cognitive rehabilitation service server measures a movable range and moving speed of a user's eye and adaptively sets a moving range and moving speed of a selection icon on a user interface according to the measured movable range and moving speed of the user's eye, wherein
the cognitive rehabilitation service server provides the rehabilitation content and then performs reassessment on the user of the user device, and wherein
a cognitive assessment result for a reference step is determined considering the reference step determined based on a correct answer rate for a set of questions provided upon the cognitive function test, and a transfer from the reference step to a relatively higher or lower step than the reference step proceeds, considering again the result of the cognitive assessment for the reference step.

2. (canceled)

3. The method of claim 1, wherein
the cognitive function test is performed on at least one of an orientation area, a memory area, an attention concentration area, a visual perception area, and a language area.

4. The method of claim 3, wherein
the cognitive function test result includes information about an assessment accuracy, an assessment time required, a user reaction time, and a score for each assessment area.

5. (canceled)

6. A cognitive rehabilitation service server performing a cognitive function rehabilitation training method, wherein
the cognitive rehabilitation service server includes a processor, wherein
the processor is configured to perform a cognitive function test,
receive a result of the cognitive function test,
determine a rehabilitation method for the cognitive function test result, and
provide rehabilitation content according to the rehabilitation method to a user device to perform rehabilitation training, wherein
the cognitive function test is performed based on touch recognition, speech recognition, or gaze tracking, wherein
the rehabilitation method is determined based on a method used for the cognitive function test, wherein
the processor detects a movement of an eye based on a gaze tracking module and tracks a position of a gaze to perform the cognitive function test and the rehabilitation training, and the processor detects the movement of the eye using the gaze tracking module and uses the detected eye movement, as a user interface, instead of a touch upon performing a cognitive function assessment and cognitive rehabilitation program, wherein
the cognitive rehabilitation service server measures a movable range and moving speed of a user's eye and adaptively sets a moving range and moving speed of a selection icon on a user interface according to the measured movable range and moving speed of the user's eye, wherein
the cognitive rehabilitation service server provides the rehabilitation content and then performs reassessment on the user of the user device, and wherein
a cognitive assessment result for a reference step is determined considering the reference step determined based on a correct answer rate for a set of questions provided upon the cognitive function test, and a transfer from the reference step to a relatively higher or lower step than the reference step proceeds, considering again the result of the cognitive assessment for the reference step.

7. (canceled)

8. The cognitive rehabilitation service server of claim 6, wherein
the cognitive function test is performed on at least one of an orientation area, a memory area, an attention concentration area, a visual perception area, and a language area.

9. The cognitive rehabilitation service server of claim 8, wherein
the cognitive function test result includes information about an assessment accuracy, an assessment time required, a user reaction time, and a score for each assessment area.

10. (canceled)
